(19) United States
(12) Patent Application Publication
(10) Pub. No.: US 2010/0180830 A1
(43) Pub. Date: Jul. 22, 2010

(54) ANIMAL LITTER AIR TREATMENT DEVICE CONTAINING ACTIVATED CARBON

(76) Inventors: Charles F. Fritter, Pleasanton, CA (US); James Siacunco, Pleasanton, CA (US)

Correspondence Address: THE CLOROX COMPANY, P.O. BOX 24305, OAKLAND, CA, 94623-1305, US

(21) Appl. No.: 12/358,237

(22) Filed: Jan. 22, 2009

(51) Int. Cl. A01K 1/01; A01K 29/00

(52) U.S. Cl. 119/500; 119/165; 119/163

(57) ABSTRACT

Disclosed herein is a portable deodorizing air treatment device adaptable for use in a litter box. The device is activated by the presence of the animal in the litter box. After the animal uses the box, the device intakes dirty air, treats it with odor-absorbing activated carbon and optionally fragrance, expelling clean unscented or scented air.

### CROSS REFERENCE TO RELATED APPLICATIONS

This application claims the benefit of U.S. Provisional Application No. 62/023,329, filed Jan. 24, 2008, which is hereby incorporated by reference in its entirety.

### FIELD OF THE INVENTION

The present invention relates generally to a portable deodorizing device to be used on or near a litter box for pets. More particularly, the present invention relates to a portable device for ingress of dirty air to be treated with air treatment materials such as activated carbon and egress of clean air after treatment.

### DESCRIPTION OF RELATED ART

Domestic, housebroken animal, particularly cats, are typically trained to urinate and defecate in a specially provided litter box. Consequently, pet owners, homeowners, veterinarians and laboratory personnel have added absorbent materials to the litter box to collect the urine and feces (i.e., animal dross). In this approach, most cats typically aid in controlling odors emanating from the box by burying their waste a short time after deposition. A major problem with this approach is that odors are not controlled during the time between deposition and burial during which time a significant plume of odor vapors are often released. Another major problem occurs with the absorbent materials themselves in that after a relatively short period of time, the dross soiled absorbent emits objectionable odors due to the presence of the animal dross.

The need to control unpleasant odors that emanate from a litter box, particularly those for a cat, has been expressed by cat owners. Animal odor control devices for animal housings exist today in several forms and varieties. For example, Walton, in U.S. Pat. No. 5,289,800 discloses a passive ventilation system. Warm air is deodorized by a charcoal filter as it rises from the animal housing and passes through the filter in the housing's roof. Griffith, in U.S. Pat. No. 4,095,559 discloses an active ventilation system. Air in the animal housing is vented away from the housing by an electric fan connected to a tubular exhaust conduit. The Griffith patent, as well as Miksitz, U.S. Pat. No. 5,044,325, and Roberts, U.S. Pat. No. 5,140,948 disclose odor control devices that are permanently fixed to the animal housing. These designs are all limited by the fact that each is specific to a specially designed and adapted animal housing.

Littermaid™ makes a fragrance-free, carbon-containing air cleaner that clamps onto a litter box and plugs into a conventional wall socket. This design is limited by the fact that the device is not activated by the animal, and is only designed for adsorption of odors using activated carbon.

What is needed is a portable device that is adaptable to any litter box that can be animal-activated so that the deodorization and other air treatments occur as soon as the animal uses the box. Thus, what is needed is a portable deodorizing device that is both effective and aesthetically pleasing to the consumer.

### SUMMARY OF THE INVENTION

An embodiment of the invention includes an air treatment device adaptable for use with a litter box comprising a housing assembly with an internal cavity. The housing has an air inlet for the ingress of “dirty air” and the inlet has downward facing slats which prevent litter material from entering the internal cavity. The internal cavity has a fan for the ingress of dirty air; a battery for powering at least the fan; activated carbon for treating the dirty air; a fragrance source for dispensing fragrance to the treated air and an outlet for the egress of treated, fragranced air. The activated carbon can be positioned either in front of or behind the fan.

Another embodiment of the invention further includes a sensor for detecting the presence of an animal and a timer which is then used to activate the device. In addition to the activated carbon filter, the device also includes at least a secondary treatment source. The secondary treatment source can be a fragrance, an odor-cancelling material and/or a sanitizing material. The secondary treatment source scents, cancels any remaining odors and/or sanitizes the carbon-filtered air. A battery is used to power the fan, the sensor, and the timer.

Air sanitization can be accomplished through the use of various sanitizers incorporated in the device described above, for example, hypochlorite or hypochlorite salts, peroxide or peroxide salts, chlorous acid or chlorous acid salts, chlorine dioxide, and/or glycols. This list is not meant to be limiting, and other suitable sanitizer materials may also be available.

### BRIEF DESCRIPTION OF THE DRAWINGS

FIG. 1 depicts the parts of an embodiment of an air treatment device.

FIG. 1A depicts the front cover of the air treatment device of FIG. 1.

FIG. 1B depicts the back cover of the air treatment device of FIG. 1.

FIG. 2 depicts the parts contained in the internal cavity of an air treatment device.

FIG. 3 depicts an air treatment device in use with a litter box.

FIG. 4A depicts an embodiment of a carbon filter cartridge.

FIG. 4B depicts an embodiment of a carbon filter cartridge combined with the front cover of the housing assembly.

### DETAILED DESCRIPTION

Before describing the present invention in detail, it is to be understood that this invention is not limited to particularly exemplified systems or process parameters as such may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an” and “the” include plural referents unless the content clearly dictates otherwise. Thus, for example, reference to a “fragrance bead” includes two or more such beads.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

The following description includes embodiments presently contemplated for carrying out the present invention. This description is made for the purpose of illustrating the general principles of the present invention and is not meant to limit the inventive concepts claimed herein.

As used herein the term “dirty” when used to describe air refers to malodor-laden air that contains odors associated with animal dross. As used herein the term “clean” when used to describe air refers to malodor-laden air, i.e., dirty air that has been passed through an odor absorbing filter, e.g., an activated carbon filter.

As used herein the term “activated carbon” means absorbent carbon-based materials, including activated and reactivated carbon-based absorbents. Activated carbon, including the material commonly called activated charcoal, is an amorphous form of carbon characterized by high adsorptivity for many gases, vapors and colloidal solids. Carbon is generally obtained by the destructive distillation of coal, wood, nut-shells, animal bones or other carbonaceous materials, including coconuts. The carbon is typically “activated” or reactivated by heating to about 800-900° C., with steam or carbon dioxide, which results in a porous internal structure.

Disclosed herein is an air treatment device for a litter box that will absorb, remove and/or contain the malodors emanating from deposited waste, especially those odors emanating from freshly deposited fecal waste that has not yet been buried by the animal. In addition, the device will expel a pleasant fragrance and/or sanitize the exiting air (as well as the air immediately surrounding the litter box).

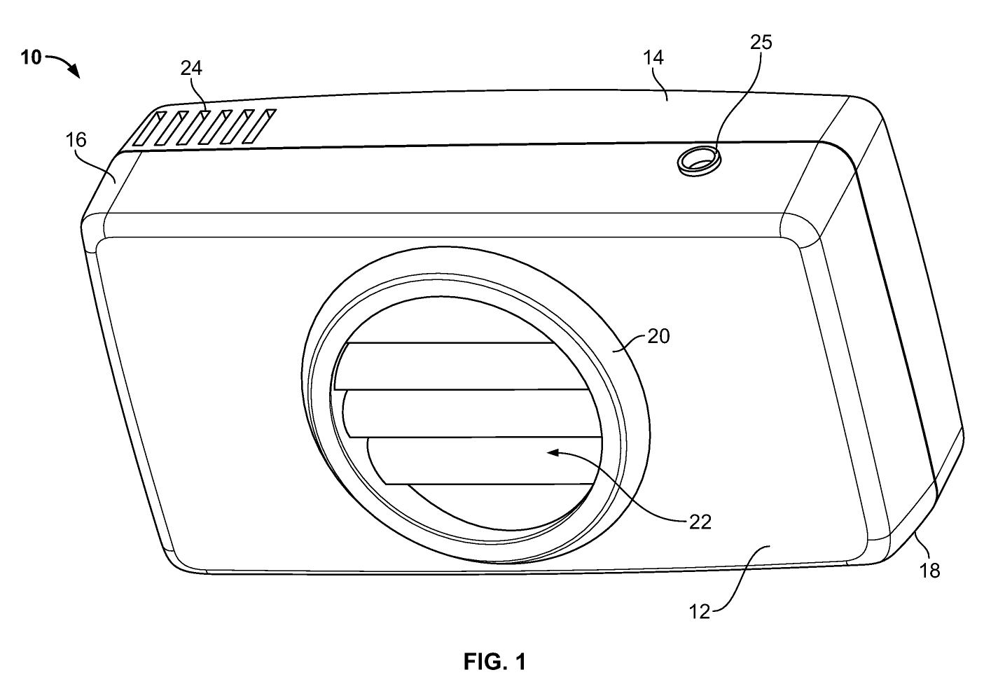

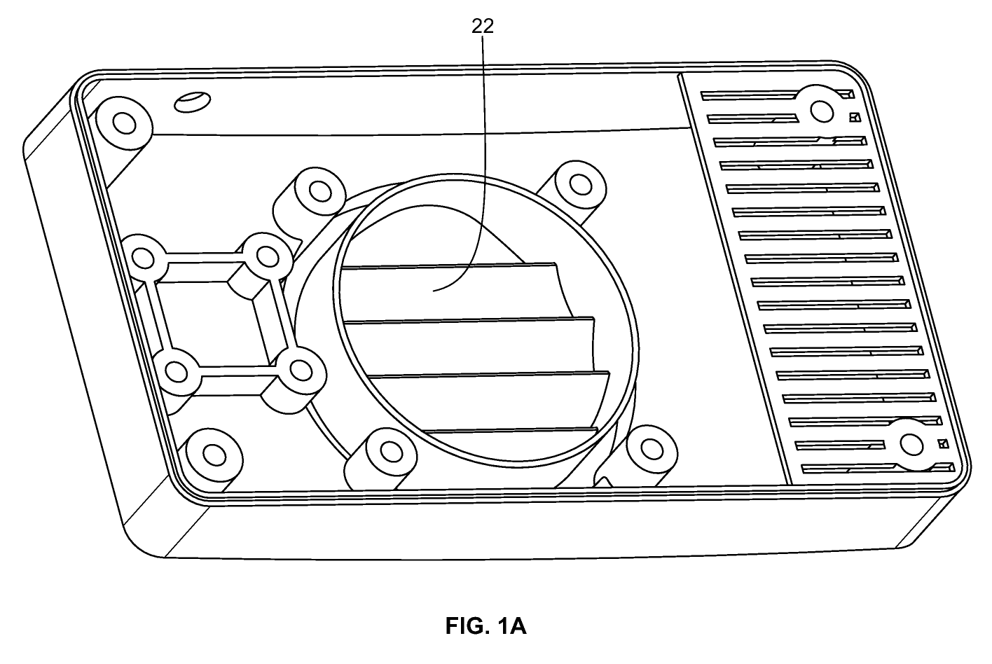

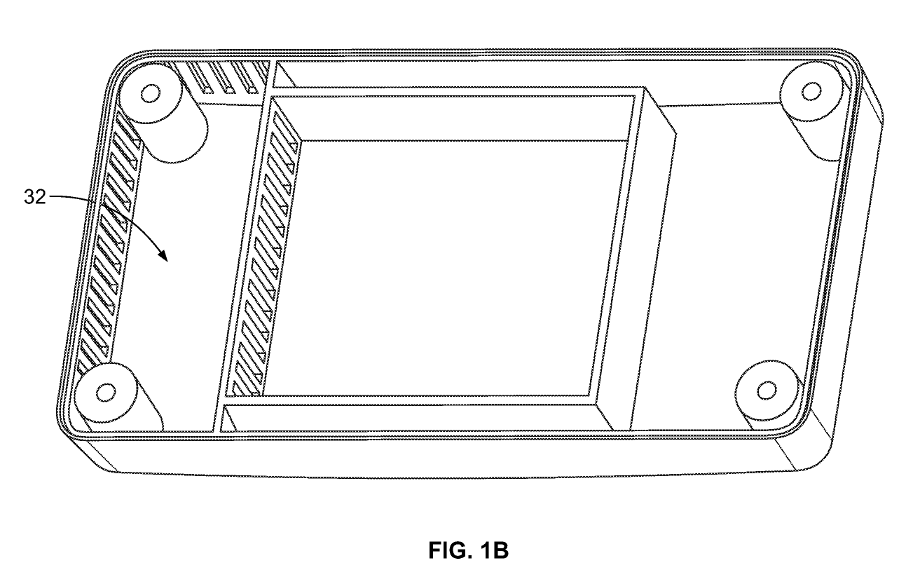

An embodiment of a compact and portable air treatment device that is capable of attachment to the side of a litter box is shown in FIG. 1. The device includes a housing assembly 10 and an interior cavity (not shown). Housing assembly 10 includes a front cover 12 and a back cover 14. When assembled the housing also includes a top 16 and a bottom 18. Front cover 12 includes an air inlet 20 having downward facing slats 22 which keep the animal litter from entering the interior of the housing. Back cover 14 includes an air outlet 24. FIG. 1A shows the interior of the front cover and FIG. 1B shows the interior of the back cover. Referring again to FIG. 1, the device may contain a sensor 25 to detect the presence of an animal and turn the fan on when an animal is detected. Additionally, the device may contain a timer (not shown) to automatically turn the fan off after a predetermined period of time, e.g. 1-30 minutes after the sensor detects that the animal is no longer present in the box. Sensor 25 may detect motion, sound, light reflection, weight or heat and should be limited in sensitivity to that corresponding to a small animal, e.g., a cat.

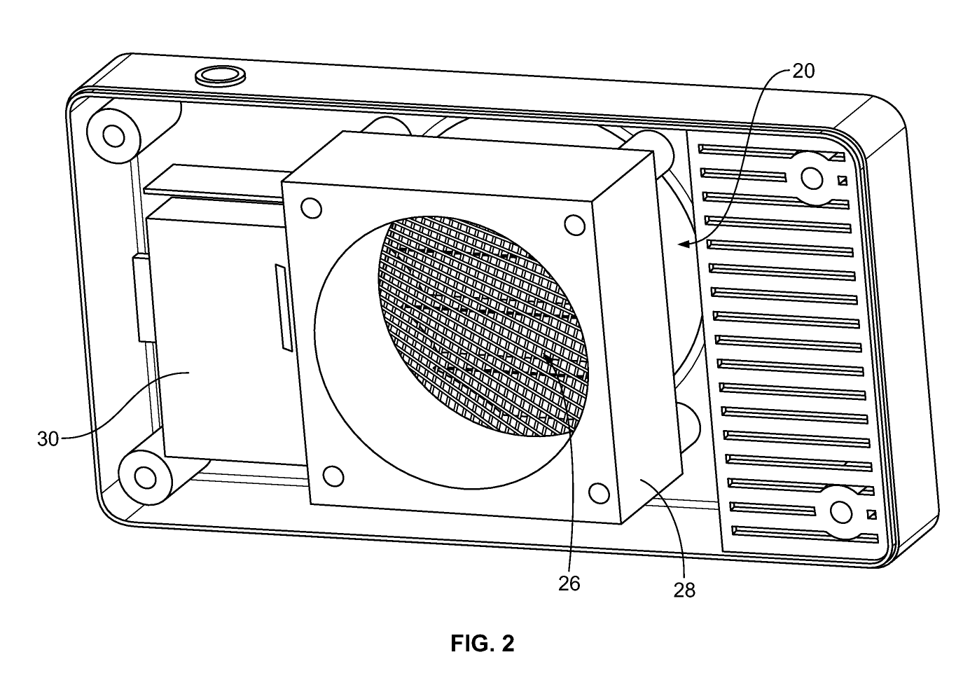

FIG. 2 shows the interior cavity of the device. An odor absorbing material 26, e.g., activated carbon, zeolite, silica gel, activated alumina, baking soda or a combination thereof is positioned between air inlet 20 and a fan 28 or alternatively between the fan and a secondary treatment material. The odor absorbing material may be in a secondary housing, e.g., a filter cartridge, or may be in a form where secondary containment is unnecessary. A battery 30, e.g., a standard 9 volt, is used to power the fan and may also be used to power sensor 25 and timer 27. Positioned between fan 28 and air outlet 24 can be a reservoir (not shown) to place a secondary treatment material such as a fragrance source to mask the odor, an air sanitizing material to sanitize the air exiting the device or an odor canceling material to neutralize the odor, e.g., a chemical(s) that reacts with an odiferous constituent contained in the waste such that any remaining odor is neutralized. Area 32 in FIG. 1B shows a possible location for the reservoir.

A preferred fragrance source comprises fragrance beads such as those supplied by commercial fragrance houses such as IFF and Firmenich. Other fragrance sources include natural materials that emit their native scents.

Odor absorbing materials in addition to activated carbon include baking soda; zeolites; activated alumina; silicas; silicates, such as diatomaceous earth metals; filter agents, such as Celatom; and aluminosilicates, such as Fuller's Earth, Montmorillonite or Bentonite, or a combination thereof.

Examples of air sanitizing and/or odor canceling materials include: acid/base effervescents; antimicrobial-impregnated polymers; sodium bicarbonate (baking soda); sodium carbonate; iron in solid or salt form; hypochlorite or hypochlorite salts, peroxide or peroxide salts, chlorous acid or chlorous acid salts, chlorine dioxide, and/or glycols.

Device 10 can be attached to the litter box in any way feasible. For example, Velcro, a clip, a clamp, a suction cup, a cloth strap with a buckle, a rubber belt, a groove(s), a slot(s) or ridge(s) compatible with a matching groove(s), slot(s) or ridge(s) on the litter box.

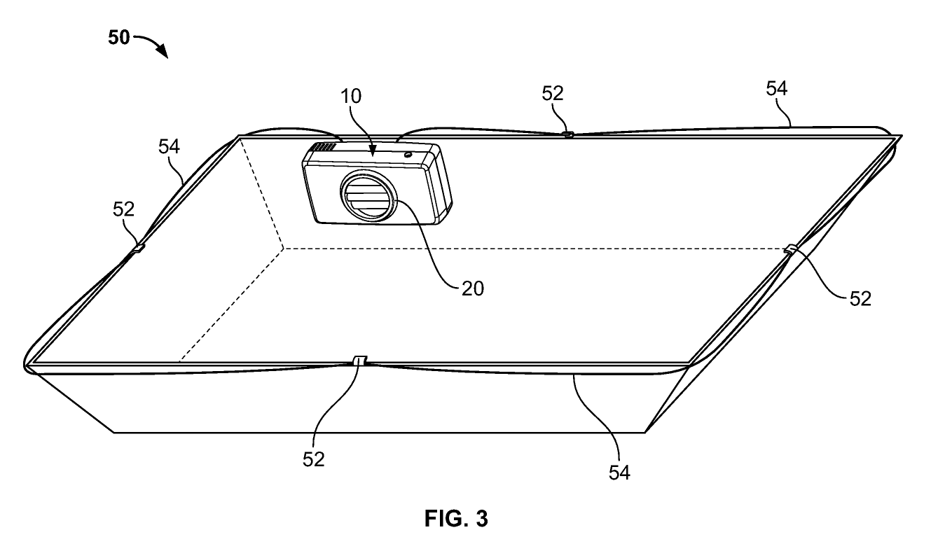

In one embodiment, air treatment device 10 can have multiple air inlet ports. FIG. 3 shows a litter box 50 with an embodiment of the air treatment device 10 of the present invention attached. Device 10 can be attached on either the interior or the exterior of the box although the interior is preferred. In this embodiment, device 10 includes several additional inlets 52 that are strategically spaced around the perimeter of the litter box, preferably on the inside of the box just below the edge of the litter box rim. Inlets 52 are in communication with the air inlet 20 of device 10 by some form of air transport mechanism, such as tubing 54. This embodiment enables malodor-laden air from in and around the litter box to be vacuumed into air treatment device 10.

The activated carbon can be in the form of a removable/replaceable cartridge. One form of carbon is carbon foam manufactured by Foamex International, Inc., which is a polyurethane foam coated with activated carbon and a binder. Substrates other than polyurethane are available such as polyester, or other similar polymers. Although activated carbon is a preferred odor absorbing material for the foam, other possible odor adsorbents could also be used.

The fragrance source and/or sanitizing material can also be in the form of a removable/replaceable cartridge or otherwise be designed to be easily removable/replaceable so that the end user can, for example, easily switch between fragrance choices and/or alternate between air sanitizing/odor canceling materials. For example, fragrance beads can be contained in packets. Depending on the type of air sanitizing/odor canceling material used, it can either be contained in packets or in a removable/replaceable cartridge.

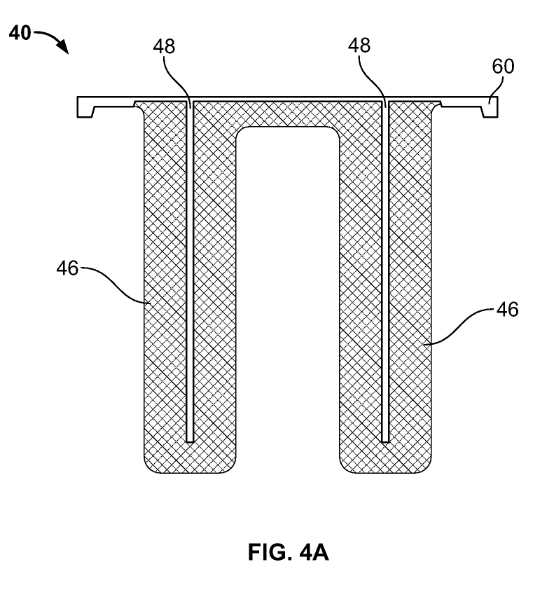

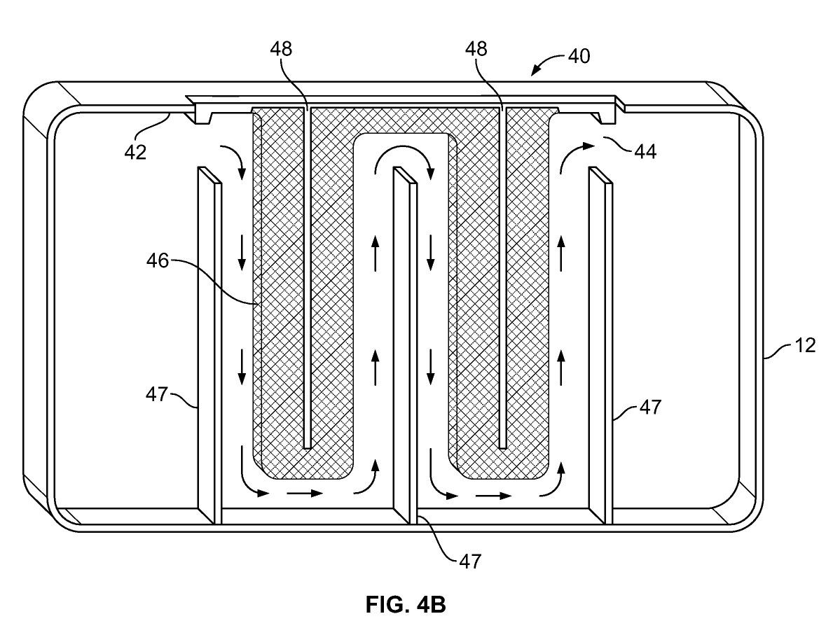

The secondary treatment material cartridge could be similar to the one shown in FIG. 4A and described below. Depending on the properties of the secondary treatment material, it may be incorporated onto a foam substrate in a manner similar to the activated carbon, i.e., a polyurethane or other suitable polymeric material can be coated with the treatment material and a binder. However, the secondary treatment material could be in any appropriate form (e.g., liquid, solid, granular, block, etc.) within the cartridge.

Referring to FIG. 4A, one embodiment of an activated carbon cartridge is shown. In this embodiment the air flow is designed to follow a convoluted, high surface area, tortuous path so that the contact time between the dirty air and the surface area of the activated carbon is maximized. The activated carbon 46 is coated with a binder onto polyurethane (i.e., “carbon foam”) and is filled along dividers 48 which protrude the top 60 of the cartridge.

Referring to FIG. 4B, cartridge 40 is designed to snap into either front cover 12 or rear cover 14 and be encompassed by dividers 47 contained on either the front or rear cover to form a sealed tortuous air flow path (front cover is shown). The front cover 12 or rear cover 14 has an air inlet 42 and an air outlet 44 which is in communication with the fan (not shown) of the device or the air inlet of the device (not shown) to force the air flow in the direction shown by the arrows.

FIGS. 4A and 4B depict 5 dividers, but any number of dividers could be utilized with the understanding that the greater the number of dividers the more tortuous the ensuing air path and the greater the contact time between the dirty air and the surface area of the activated carbon. Thus, the carbon filter can effectively utilize a very small physical area if used together with a highly tortuous air flow path.

Filter cartridge 40 can be plastic and can be manufactured using well known plastics fabrication techniques, such as injection molding and thermoforming. It should be designed to “snap fit” securely into either the front or rear cover of the housing assembly.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

We claim:

1. An air treatment device adaptable for use with a litter box comprising:
a housing assembly having a front, a back, a top, a bottom and an internal cavity;
said front having an air inlet for the ingress of dirty air, said inlet having downward facing slats which prevent litter material from entering the internal cavity;
said internal cavity having a fan for the ingress of dirty air; a battery for powering at least the fan; activated carbon for treating the dirty air;
said top having an outlet for the egress of treated air; wherein
the air inlet is in communication with the activated carbon; the activated carbon is in communication with the fan and the fan is in communication with the air outlet.

2. The air treatment device recited in claim 1, wherein the dirty air follows a tortuous path from the inlet through the activated carbon to the outlet to maximize the contact of the dirty air with the surface area of the activated carbon.

3. The air treatment device recited in claim 1, further comprising a fragrance source, wherein the fan is in communication with the fragrance source and the fragrance source is in communication with the air outlet.

4. The air treatment device recited in claim 3, wherein the fragrance source is fragrance beads.

5. The air treatment device recited in claim 1, further comprising an air sanitizing material, wherein the fan is in communication with the air sanitizing material and the air sanitizing material is in communication with the air outlet.

6. The air treatment device recited in claim 5, wherein the air sanitizing material is hypochlorite, hypochlorite salts, peroxide, peroxide salts, chlorous acid, chlorous acid salts, chlorine dioxide, glycols, antimicrobial-impregnated polymers or combinations thereof.

7. The air treatment device recited in claim 1, wherein the activated carbon is carbon foam, granulated carbon, a carbon block, and combinations thereof.

8. The air treatment device recited in claim 1, further comprising air intake ports in communication with the air inlet, wherein said air intake ports are located around the internal perimeter of the litter box.

9. The air treatment device recited in claim 1, further comprising an attachment component for attaching the device to the litter box.

10. The air treatment device recited in claim 9, wherein the attachment component is Velcro, a clip, a clamp, a suction cup, a cloth strap with a buckle, a rubber belt, a groove, slot or ridge compatible with a matching groove, slot or ridge on the litter box.

11. The air treatment device recited in claim 1, further comprising a sensor for detecting the presence of an animal and timer, wherein the sensor and timer are in communication with the fan such that the fan is turned on when an animal is detected and the fan is turned off a predetermined amount of time after the initial detection of the animal.

12. The air treatment device recited in claim 11, wherein the sensor detects heat, motion or sound.

13. The air treatment device recited in claim 11, wherein the battery further powers the sensor and timer.

14. The air treatment device recited in claim 11, wherein the predetermined amount of time is adjustable.

15. An air treatment device adaptable for use with a litter box comprising:
a housing assembly having a front, a back, a top, a bottom and an internal cavity;
said front having an air inlet for the ingress of dirty air, said inlet having downward facing slats which prevent litter material from entering the internal cavity;
said housing further having a sensor for detecting the presence of an animal;
said internal cavity having a fan for the ingress of dirty air; activated carbon for treating the dirty air and expelling filtered air; a fragrance source and/or a sanitizing material for scenting and/or sanitizing the filtered air; a battery for powering at least the fan, the sensor, and the timer;
said top having an outlet for the egress of treated air; wherein the air inlet is in communication with the activated carbon; the activated carbon is in communication with the fan, the fan is in communication with the fragrance source and/or the sanitizing material and the fragrance source and/or sanitizing material is in communication with the air outlet; and
wherein said fan is additionally in communication with said sensor and said timer.
